(19) United States
(12) Patent Application Publication
(10) Pub. No.: US 2026/0346745 A1
(43) Pub. Date: Nov. 5, 2026

(54) Automated Battery Charging and Power Management for Perioperative Cobotic Systems

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/432,199

(22) Filed: Dec. 24, 2025

Related U.S. Application Data
(63) Continuation-in-part of application No. 19/195,731, filed on Apr. 30, 2025.

(51) Int. Cl. B60L 53/31; A61B 34/32; A61B 90/00; B60L 53/16; B60L 53/30; B60L 53/36; B60L 53/62; B60L 53/65; B60L 53/66; B60L 53/67; B60L 53/80; B60L 58/12; B60L 58/16; G05D 1/43; G05D 1/622; G05D 1/661; G08B 21/18

(52) U.S. Cl. CPC B60L 53/31; A61B 34/32; A61B 90/08; B60L 53/16; B60L 53/305; B60L 53/36; B60L 53/62; B60L 53/65; B60L 53/66; B60L 53/67; B60L 53/80; B60L 58/12; B60L 58/16; G05D 1/43; G05D 1/637; G05D 1/661; G08B 21/182; A61B 2090/0807

(57) ABSTRACT

A perioperative cobotic system is provided comprising a mobile robotic platform equipped with an integrated power management system and autonomous charging capability. The system monitors battery state-of-charge, temperature, and power consumption in real time, and dynamically regulates power distribution across robotic subsystems. The platform autonomously navigates to a docking station using environment mapping, obstacle avoidance, and sterile-field compliance protocols. A sterility-compliant charging interface facilitates safe electrical coupling outside sterile zones. The system supports adaptive charging protocols based on battery chemistry, thermal profile, and operational urgency. A fleet coordination engine manages charging schedules across multiple units to optimize power availability and facility load. Redundant battery architecture with hot-swap capability allows uninterrupted operation. A predictive analytics engine forecasts energy demand and battery health, while a user interface provides real-time diagnostics, alerts, and manual overrides.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by reference.

### TECHNICAL FIELD

The present disclosure relates generally to robotic systems for surgical environments, and more specifically to automated battery charging and intelligent power management systems for mobile perioperative cobots operating within sterile or semi-sterile hospital settings.

### BACKGROUND

Mobile robotic systems are increasingly being adopted in surgical and perioperative environments to support a range of critical functions including instrument delivery, intraoperative sensing, workflow coordination, and surgical team collaboration. These so-called “cobots” are designed to operate in close proximity to clinical personnel and sterile fields, often moving autonomously through dynamic and spatially constrained operating rooms (ORs) and adjacent zones. As such, their uninterrupted availability, safe maneuverability, and predictable performance are essential for maintaining surgical efficiency and patient safety.

A key limiting factor for the operational reliability of such cobots is their dependence on onboard battery systems, which must balance high energy density with rigorous safety, hygiene, and mobility requirements. Existing mobile robotic platforms often rely on manual charging workflows, static recharging stations, or inflexible power distribution schemes, all of which introduce undesirable latency, human intervention, and scheduling conflicts in high-demand clinical environments. Moreover, current approaches to battery management rarely account for the specific energy needs of surgical tasks, nor do they dynamically adapt charging protocols based on evolving battery conditions or facility-wide power constraints.

Charging infrastructure itself presents additional design challenges in surgical settings. Conventional robotic docking stations are typically not designed for compliance with OR sterility protocols. Unshielded contacts, exposed cabling, and unconstrained charging zones may inadvertently breach sterile fields or introduce contamination risks. Further, the absence of predictive power analytics, load-balancing coordination across multiple units, or real-time OR system integration limits the utility of current power management solutions in high-acuity settings.

Accordingly, there is a need for a perioperative cobotic platform that incorporates intelligent, autonomous, and sterility-compliant battery charging and power management systems. Such a platform would ideally support real-time power regulation across robotic subsystems, self-directed navigation to charging infrastructure, adaptive charging protocols, predictive energy analytics, and coordinated fleet-level scheduling, all while maintaining operational integrity within OR sterility boundaries.

### SUMMARY

The present disclosure relates to a perioperative cobotic system incorporating an integrated battery charging and power management architecture. In one embodiment, the system includes a mobile robotic platform configured for autonomous operation in surgical and perioperative environments. The robotic platform comprises an onboard battery and a power management system configured to monitor real-time battery parameters, including charge level, temperature, discharge rate, and task-specific power demands, and to regulate power distribution among the platform's subsystems to optimize operational performance and runtime.

The system further includes an autonomous charging navigation module configured to detect power thresholds, map the clinical environment, and navigate the robotic platform to a designated docking station while avoiding obstacles and maintaining compliance with sterile field boundaries. Upon reaching the docking station, a sterility-compliant charging interface engages with the robotic platform to initiate charging without breaching sterility constraints.

In certain embodiments, the docking station includes bidirectional communication capabilities and supports dynamic charging initiation, progress monitoring, and termination based on real-time power diagnostics. The station may include multiple charging ports to support sequential or concurrent charging of multiple mobile robotic units or battery packs.

Charging operations are governed by adaptive protocols that modulate voltage and current levels based on battery chemistry, thermal profile, remaining cycle life, and projected usage. The system may further include redundant or hot-swappable battery configurations with authentication and safety interlocks to support continuous operation during long-duration procedures.

Fleet-level coordination is provided via a scheduling module that prioritizes charging readiness across multiple robotic units, mitigates docking congestion, and balances facility power consumption. A predictive power analytics engine leverages machine learning to forecast energy consumption, identify degradation trends, and generate alerts related to battery health or procedural readiness. A user-facing interface provides real-time dashboards, battery diagnostics, and override capabilities for clinical staff.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a schematic diagram of a perioperative cobotic system comprising a mobile robotic platform and an intelligent docking station.

FIG. 2 illustrates a block diagram of the integrated power management system and its connections to the cobot’s subsystems.

FIG. 3 depicts the autonomous navigation process of the mobile robotic platform as it approaches a designated charging station while avoiding obstacles and respecting sterile field boundaries.

FIG. 4 shows a perspective view of a sterility-compliant charging.

FIG. 5 is a flow chart depicting a method for adaptive charging protocol selection.

FIG. 6 is a block diagram showing fleet-level coordination among multiple cobots.

FIG. 7 is a schematic of a dual-battery architecture.

FIG. 8 is a flow diagram illustrating predictive power analytics for task-based consumption forecasting and maintenance alert generation.

FIG. 9 is a diagram of a user interface display.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

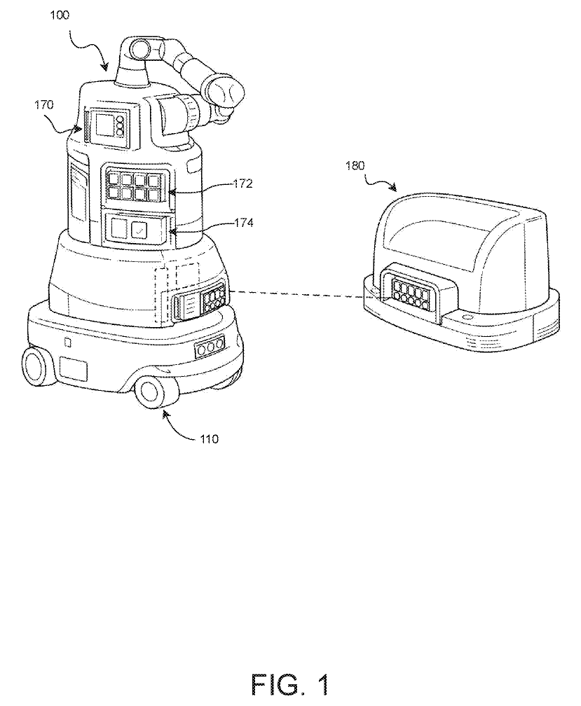

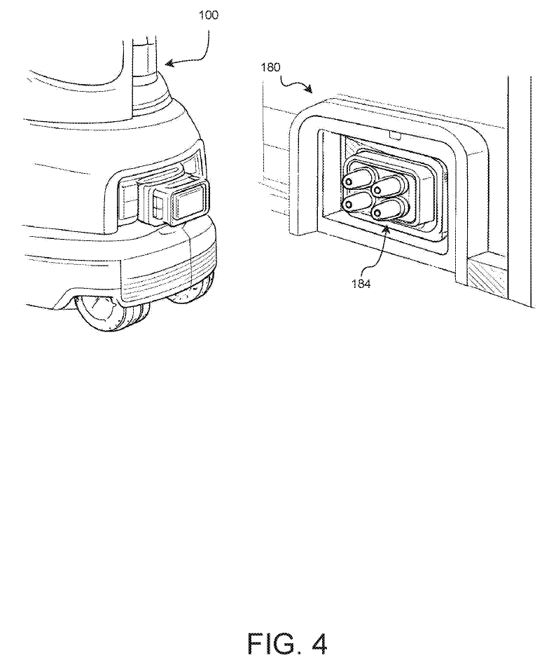

Referring now to FIG. 1, a perioperative cobotic system 100 is depicted in relation to an automated docking station 180. The cobot (collaborative robot) includes a mobile base 110 that houses a rechargeable battery pack and associated power circuitry. In the illustrated embodiment, the base 110 serves as the platform providing mobility and also contains the control system 170 (including processors, memory, and communication hardware) and an integrated power management unit 172. The power management unit monitors the battery’s state and distributes power to all cobot subsystems, including drive motors, sensors, and robotic actuators. The mobile base 110 may include ports or connectors for recharging the battery pack, which interface with the docking station 180. The docking station 180 is positioned at a convenient location (for example, at the periphery of the operating room or in a non-sterile corridor) to allow the cobot 100 to autonomously dock for charging when needed. During normal operation, the cobot 100 can undock and move freely; when battery levels fall below a threshold or a charging opportunity is scheduled (e.g., between surgeries), the cobot navigates to the docking station 180 to recharge. In some use cases, the cobot may dock during off-duty hours (such as overnight) for full recharging and system updates or data upload. The docking station 180 provides a stable platform and guided alignment features to ensure reliable electrical coupling with the cobot’s charging interface as will be described in detail with FIG. 4. Bi-directional communication between the cobot 100 and docking station 180 allows the station to authenticate the cobot, verify proper alignment, and initiate a controlled charging session.

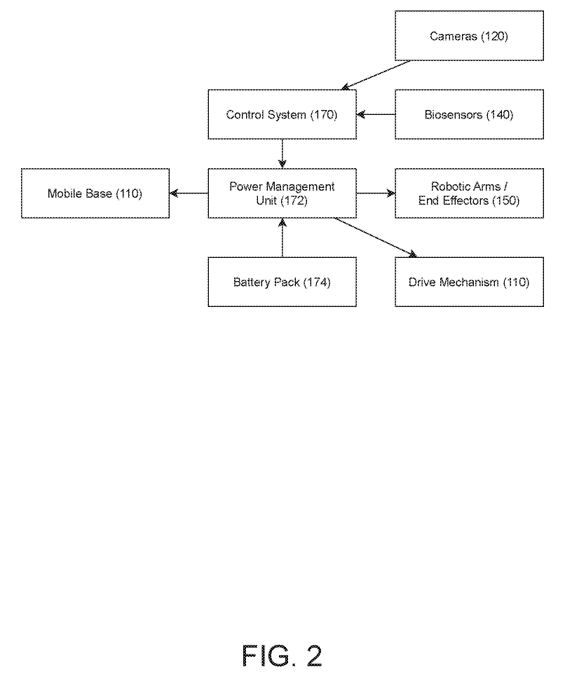

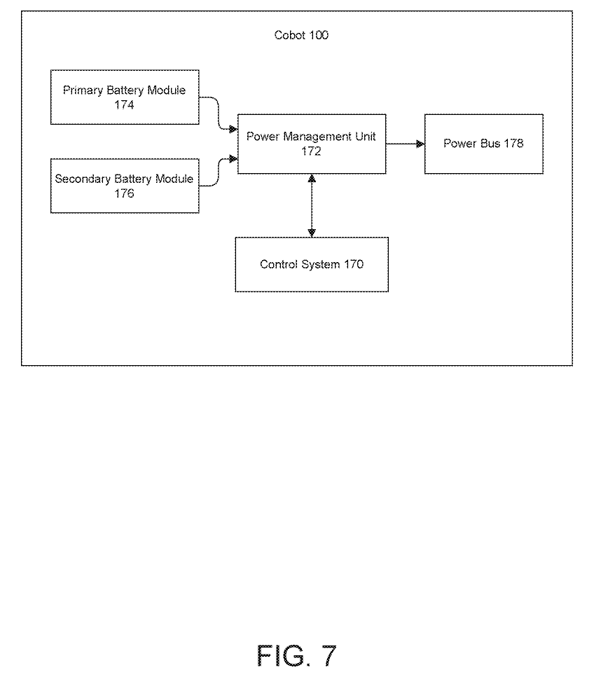

FIG. 2 illustrates a block diagram of the cobot’s integrated power management system, which is responsible for monitoring and distributing power throughout the cobot 100. At the center of this system is the power management unit 172, which may be implemented as part of the control system 170 or as a dedicated battery management controller. The power management unit 172 is electrically connected to a main battery pack 174 (and in certain embodiments, a secondary battery pack as discussed with FIG. 7) and to all major subsystems of the cobot. These subsystems include the drive mechanism (motors and locomotion components in the base 110), the sensor suite (e.g., cameras 120, LiDAR, biosensors 140), the robotic arms or end effectors 150, and the communication and computing modules. The power management unit 172 continually monitors battery 174 parameters such as state-of-charge (SoC), voltage, current draw, and temperature. It also tracks the power consumption of each subsystem in real time. Using this information, the unit 172 actively regulates power distribution to optimize performance and conserve energy. For instance, if one subsystem (such as locomotion) temporarily requires a surge of power, the unit can allocate additional current to that subsystem while ensuring critical systems (like control and sensors) remain powered. Conversely, during periods of low activity, the power management system may scale down or idle certain components to extend the operational time of the cobot. The block diagram of FIG. 2 shows feedback lines from the subsystems to the power management unit 172, representing the sensing of power usage and health metrics. Additionally, safety circuits are included to protect against over-voltage, over-current, or overheating conditions – for example, if the battery temperature exceeds a threshold, the power management unit may reduce charging rate or temporarily shut down non-essential functions to prevent damage. By integrating sensing and control logic, the power management system ensures the cobot can operate for an entire procedure (often targeting 8–12 hours on a full charge) and gracefully manage power to avoid sudden outages. The inclusion of power management circuitry and charging interfaces in the base allows the battery 174 to be recharged without removal, simply by connecting to an external power source via the docking station or another plug-in connection.

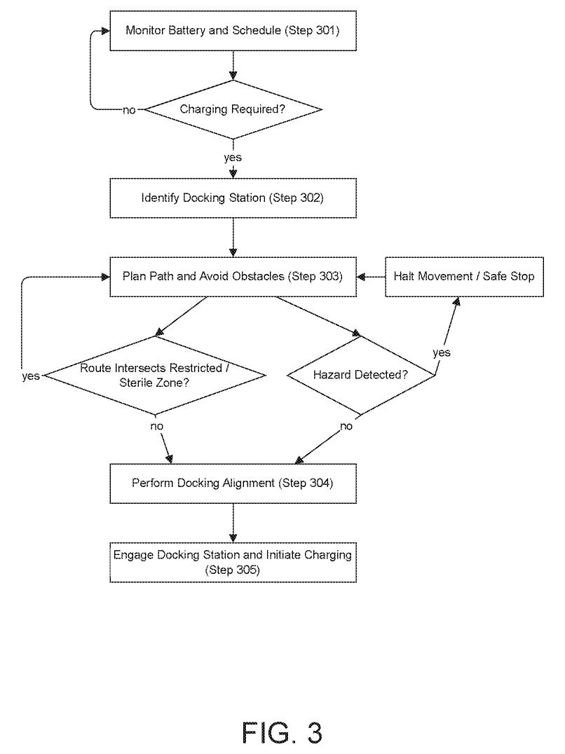

FIG. 3 is a flowchart illustrating an autonomous charging navigation process executed by the cobot’s control system 170, and more particularly by an autonomous charging navigation module thereof. The flowchart describes a sequence of steps by which the cobot 100 detects a need for battery charging and autonomously navigates to a docking station 180 without human intervention.

In Step 301 (Monitor Battery and Schedule), the cobot 100 continuously monitors one or more battery parameters, including state-of-charge, and optionally accesses a surgical or operational schedule. When the battery state-of-charge falls below a predefined threshold, or when a scheduled charging opportunity is detected (for example, a break between procedures), the control system 170 determines that charging is required. In some embodiments, upcoming task duration or projected power demand is evaluated, such that the cobot proactively initiates a charging routine when a forthcoming task is expected to exceed available battery capacity.

In Step 302 (Identify Docking Station), the autonomous charging navigation module identifies a location of an appropriate docking station 180. This step may include retrieving a stored map of the clinical environment, identifying a previously mapped docking location, or detecting visual, infrared, or electromagnetic fiducial markers associated with the docking station. Sensor data from cameras, LiDAR, or other perception systems is used to confirm station position and availability.

In Step 303 (Plan Path and Avoid Obstacles), the control system 170 generates a navigation path from a current cobot location to the identified docking station 180. The planned path avoids obstacles, equipment, personnel, and defined sterile zones within the operating room or adjacent areas. During traversal, the cobot employs real-time obstacle detection and avoidance techniques, including dynamic trajectory adjustment or temporary pausing when a moving object or person is detected. Sterile-field avoidance rules are enforced throughout path execution.

In Step 304 (Perform Docking Alignment), as the cobot 100 approaches the docking station 180, the navigation module transitions the cobot into a fine positioning mode. Using precision sensors such as cameras or alignment lasers, the cobot aligns its charging interface with a corresponding connector of the docking station. Visual fiducials, alignment guides, tapered surfaces, or magnetic alignment aids may be used to achieve accurate orientation and positioning prior to engagement.

In Step 305 (Engage Docking Station and Initiate Charging), the cobot advances until its charging port physically engages the docking station 180. The system verifies successful docking by detecting electrical contact or receiving a confirmation signal from the docking station. Upon verification, the control system 170 establishes communication with the docking station and initiates a charging sequence. Charging current is delivered to the battery 174 under supervision of the power management unit 172. During charging, selected drive or mobility functions of the cobot may be powered down. Upon completion of charging, or upon reaching a sufficient charge level for a next task, the cobot disengages from the docking station and resumes normal operation.

Throughout Steps 301–305, safety monitoring is continuously performed. Emergency stop functionality remains active, and if a hazard is detected at any point, the cobot halts movement to prevent collision or contamination. If a required navigation route intersects a restricted or active sterile zone, the control system either delays navigation or computes an alternate path. The autonomous charging process illustrated in FIG. 3 thereby enables the cobot fleet to manage power replenishment in a safe, efficient, and sterility-compliant manner, reducing downtime and manual intervention in perioperative environments.

FIG. 4 provides a detailed perspective view of the cobot’s sterility-compliant charging interface and the corresponding features of the docking station 180. In order to function within or near sterile surgical environments, the charging interface is specially designed to maintain cleanliness and avoid contamination of sterile fields. Key aspects of the charging interface include:

Sealed Connectors: The electrical contacts 184 on both the cobot and the docking station are enclosed in a sealed housing or recess. When the cobot is undocked, a protective cover or flush-mounted door keeps the cobot’s charging port closed, preventing ingress of fluids or contaminants. Upon docking, this cover automatically opens (or is pushed aside by the station connector) to expose the contacts. All contact surfaces are made of sterilizable materials (e.g., medical-grade stainless steel or coated copper) and are designed to be easily cleaned.

Guided Mating Surfaces: The interface employs mechanical guiding features that ensure proper alignment without precise human assistance. For example, the docking station 180 may have conical or funnel-like guide features and male connector prongs that fit into corresponding female receptacles on the cobot. These guides can have curved, concave surfaces that self-center the cobot as it docks, compensating for minor misalignments. Magnets or magnetic pads can be used to gently pull the cobot’s port into the final position and hold it during charging. This not only streamlines the docking process but also reduces wear from repeated connections.

Sterile Barrier or Alcove: In some embodiments, the charging interface is located in an isolated alcove or behind a sterile barrier. FIG. 4 shows an example where the docking station 180 is built into a wall or equipment bay that has a sterile side and a non-sterile side. The cobot 100 approaches the non-sterile side to dock, while the power transfer occurs through a sealed wall interface, preventing any airflow or particle transfer into the sterile surgical area. In other embodiments, the cobot itself may feature a self-disinfecting connector – for instance, UV-C LEDs or antimicrobial coatings around the port that activate after use to neutralize any contaminants.

Robust, Quick-Connect Mechanism: The charging connectors are designed to enable rapid engagement and disengagement. A spring-loaded or lever mechanism may ensure that once the cobot is in place, the electrical contacts press firmly together to achieve a low-resistance connection. Conversely, if the cobot needs to undock quickly (e.g., to handle an emergency task), the mechanism releases without undue sticking. Importantly, the materials chosen (plastics, metals, insulation) can withstand frequent sterilization cycles, cleaning with hospital disinfectants, and high-temperatures if necessary (for example, if parts of the connector are removable for autoclaving).

Indicator and Safety Features: The interface includes visual or electronic indicators to show the status of the connection. An LED on the station might illuminate when a successful dock is made and power transfer is active, reducing guesswork for staff. Additionally, interlock sensors prevent electrical current from flowing unless a proper connection is confirmed (to avoid arcing or exposed live contacts). For instance, a small switch can detect the physical presence of the cobot’s plug, and only then will the station 180 supply power. This ensures that staff cannot accidentally contact live prongs and that the cobot is not energized in a way that could create sparks in an oxygen-rich surgical environment.

Through these features, the charging interface of FIG. 4 is sterility-compliant, meaning it does not compromise the aseptic conditions of the operating room. The cobot only charges in designated safe zones or via interfaces that isolate any non-sterile components, in accordance with hospital infection control protocols. This design allows the cobot to be recharged even between or during procedures (if it can remain near the OR) without introducing contamination, thus greatly enhancing its usability in perioperative settings.

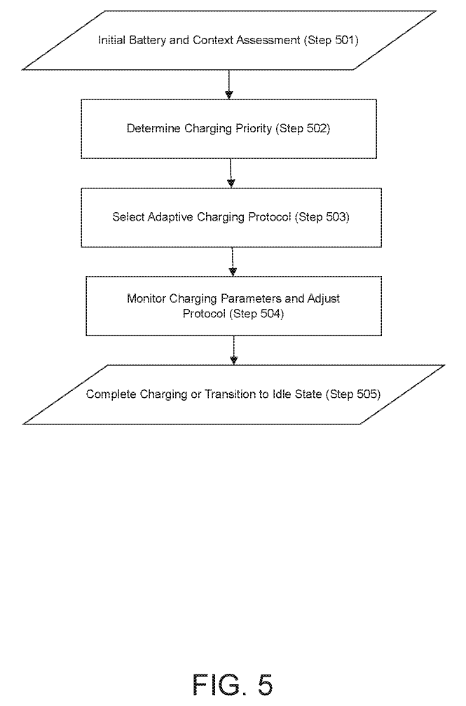

FIG. 5 is a flowchart illustrating an adaptive charging protocol selection process executed by the cobot’s power management system 172 when the cobot 100 is connected to a charging source, such as the docking station 180. The flowchart defines a sequence of steps by which charging parameters are dynamically selected and adjusted based on battery condition, operational urgency, and projected usage.

Initial Battery and Context Assessment (Step 501): Upon docking and initiation of a charging session, the power management unit 172 reads one or more current parameters of the battery 174, including state-of-charge, battery temperature, and battery health metrics such as cycle count and capacity degradation. The system further retrieves contextual data indicative of anticipated usage, including a scheduled time until a next task, procedure, or deployment of the cobot 100.

Determine Charging Priority (Step 502): Based on the assessed battery parameters and retrieved contextual data, the power management system determines a charging priority level. If the battery state-of-charge is critically low or the cobot 100 is scheduled for imminent redeployment, the system assigns a high-priority charging mode. If sufficient idle time is available or the battery is only moderately depleted, the system assigns a standard or low-priority charging mode optimized for battery longevity.

Select Adaptive Charging Protocol (Step 503): In response to the determined charging priority, the control system selects an appropriate charging protocol from a plurality of predefined charging profiles. Each charging profile defines voltage and current characteristics over time. For example, a high-priority profile may implement a constant-current/constant-voltage (CC-CV) fast-charging sequence, while a lower-priority profile may implement a maintenance or trickle-charging sequence. The selection further accounts for battery chemistry such that charging curves are tailored for lithium-ion, nickel-metal hydride, or other battery types.

Monitor Charging Parameters and Adjust Protocol (Step 504): During execution of the selected charging protocol, the power management system continuously monitors charging parameters including battery voltage, charging current, and battery temperature. If monitored conditions indicate deviation from predefined safety thresholds, the system dynamically modifies the charging protocol, including reducing charging current, transitioning to a thermal regulation state, temporarily pausing charging, or reverting to a safe fallback mode.

Complete Charging or Transition to Idle State (Step 505): The charging process concludes when the battery 174 reaches a target charge level or when deployment of the cobot 100 is required prior to full charge completion. If full charge is achieved and the cobot remains docked, the system transitions to a maintenance or trickle-charge mode to sustain battery readiness without overcharging. If charging is terminated early due to scheduling constraints, the cobot undocks with sufficient charge to perform its next assigned task.

The adaptive charging process illustrated in FIG. 5 thereby balances rapid operational readiness with long-term battery health. By dynamically selecting and adjusting charging protocols based on real-time battery data and anticipated usage, the system enables fast charging when clinically necessary while favoring gentler charging whenever possible. All charging decisions are performed autonomously without human intervention, reducing error and maximizing uptime across the cobot fleet.

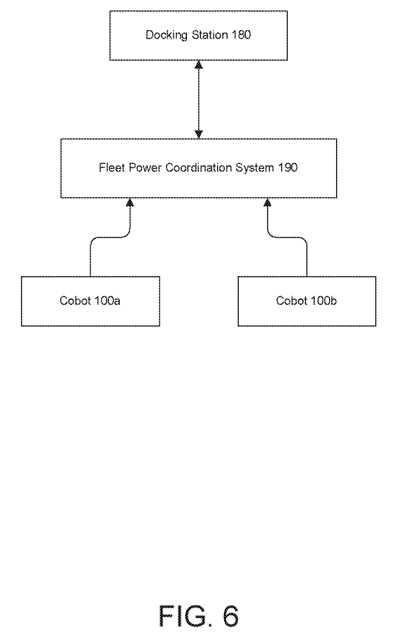

FIG. 6 is a block diagram illustrating fleet-level power coordination for a plurality of perioperative cobots operating within a facility. The block diagram represents functional relationships among multiple mobile robotic devices (cobots 100, 100a, 100b), one or more docking stations 180, and a fleet power coordination system 190 that collectively manage charging operations and facility power resources.

As shown in FIG. 6, each cobot 100 includes a communication interface configured to transmit operational status data, including battery state-of-charge, predicted energy demand, and charging requests, to the fleet power coordination system 190 over a secure wired or wireless network. The fleet power coordination system 190 may be implemented as a centralized server, a cloud-based service, or a distributed control module executing cooperative coordination logic across the cobot fleet.

The fleet power coordination system 190 includes a charging scheduling module configured to manage charging slot assignments for a plurality of docking stations 180. Upon receiving a charging request from a cobot, the scheduling module evaluates docking station availability and assigns a charging station identifier and time slot to the requesting cobot. This prevents simultaneous docking conflicts and ensures that no two cobots attempt to access the same docking station at the same time.

The fleet power coordination system 190 further includes a prioritization module configured to rank charging requests based on one or more urgency criteria, including battery level, upcoming procedural assignments, projected task duration, and clinical criticality of the cobot. Higher-priority cobots are granted earlier or exclusive access to charging resources, while lower-priority cobots may be instructed to remain in a standby or delayed-charging state.

FIG. 6 further illustrates a load-balancing module within the fleet power coordination system 190. The load-balancing module monitors aggregate power demand across the docking stations 180 and coordinates charging operations to limit peak electrical load on the facility power infrastructure. Charging sessions may be staggered, throttled, or temporarily delayed to prevent excessive current draw or demand spikes, particularly during peak facility usage periods.

Each docking station 180 is represented in FIG. 6 as a functional block including a charging interface and a communication module configured to report station status, occupancy, and charging progress to the fleet power coordination system 190. The fleet power coordination system uses this information to dynamically update scheduling decisions and reassign cobots to alternate docking stations when necessary.

FIG. 6 further includes a fleet monitoring and user interface block operatively coupled to the fleet power coordination system 190. The fleet monitoring interface provides real-time visibility into cobot battery states, charging assignments, docking station utilization, and predicted readiness. In certain embodiments, the interface enables authorized personnel to manually override automated scheduling decisions.

In addition to centralized coordination, FIG. 6 illustrates optional peer-to-peer communication links among cobots 100, enabling cooperative behavior such as local negotiation of charging order, sharing of navigation or congestion data, and coordination of approach paths to assigned docking stations. Such peer-to-peer coordination operates under constraints or policies issued by the fleet power coordination system 190.

Through the functional blocks and communication pathways shown in FIG. 6, the system achieves coordinated fleet-level charging management that maximizes cobot availability, minimizes charging congestion, and reduces stress on facility electrical infrastructure. The block diagram representation emphasizes logical control relationships and data flow among system components rather than physical layout, and supports scalable deployment across multiple cobots and docking stations within a perioperative environment.

FIG. 7 illustrates a redundant battery architecture for the cobot 100, featuring dual battery packs arranged to allow hot-swapping without interrupting the cobot’s operation. In this embodiment, the cobot is equipped with a primary battery module 174 and a secondary battery module 176, both connected to the power bus 178 that supplies the various subsystems. The architecture is designed such that either battery alone can power the cobot if the other is removed or depleted, thereby providing redundancy. This is particularly valuable for long surgical procedures or continuous usage scenarios where the cobot cannot be powered down.

In the schematic of FIG. 7, both battery modules 174 and 176 are plugged into a power management circuit (which could be part of unit 172). Diode or MOSFET-based isolation may be used so that each battery can feed the system but prevents backflow into a removed battery. For example, each battery has a control switch that automatically disconnects it when removed, and the remaining battery immediately takes up the load. A hot-swap controller IC can manage this transition, ensuring there is no drop in the supply voltage when one battery is pulled out. The power management unit also balances the discharge between both batteries when both are present, and can charge both (either sequentially or in parallel with appropriate circuitry) when the cobot is docked.

To safely enable hot-swapping, interlock mechanisms are implemented. Physically, each battery module 174, 176 may be accessible via a quick-release slot on the cobot’s base or torso. An interlock switch or sensor on each battery slot detects if the battery is present and latched. The cobot’s control system 170 only allows one battery to be unlocked at a time. For instance, if a technician needs to replace a battery during operation, they would command the cobot (via the UI or a hardware button) to enter a hot-swap mode. The system then might display or announce which battery (module A or B) can be safely removed. Only that module’s latch will disengage while the other battery keeps the system powered. Once the first battery is removed and replaced with a fresh module, the cobot can switch and allow the second one to be swapped if needed. This sequencing prevents the scenario of both batteries being removed simultaneously (which would cut power). Additionally, the interlock logic ensures that the new battery is authenticated and functioning before transferring load to it. Each battery module can contain a small memory or ID chip that the cobot reads upon insertion – this authentication step verifies that the battery is of the correct type, not faulty or past its service life, and even logs the insertion event for maintenance tracking.

During hot-swap, the power management unit 172 may momentarily draw power from a large capacitor or auxiliary power source to smooth out the transition, though typically one battery is always online so there is no interruption. The redundant architecture also means the cobot can endure a battery failure: if one pack fails or becomes depleted unexpectedly, the other pack automatically continues supplying power so the cobot can safely complete its task or at least move to a safe state or docking station.

Notably, FIG. 7’s design aligns with known hot-swappable power systems in robotics, where a hot-swappable power module is coupled to a primary energy bus of the robot. Here, each battery module 174, 176 can be considered a hot-swappable power module, and the cobot’s frame provides a receiver for these modules. The advantage of this approach is uninterrupted operation – the cobot need not leave the surgical floor or stop its activity for a lengthy recharge; instead, a fresh battery can be brought to it (or it can quickly swap at a battery change station) and continue working. This is especially important in critical healthcare environments where downtime could impact patient care.

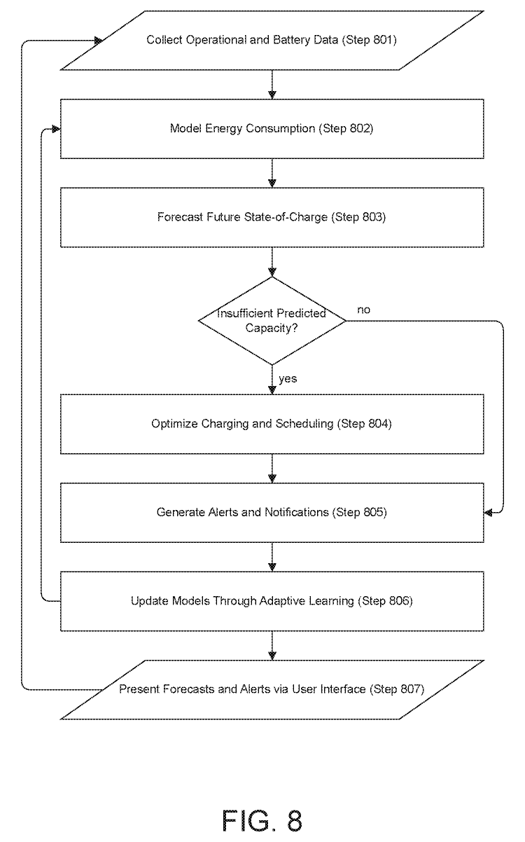

The system also incorporates tracking and alerts for battery health as part of the interlock mechanism. Each insertion or removal is logged by the control system. If a battery module has been in service too long or shows signs of degradation (through the analytics engine of FIG. 8), the system can alert technicians to replace it at the next opportunity. In summary, FIG. 7 demonstrates a robust power supply architecture for the cobot that emphasizes redundancy, safety, and continuous availability through hot-swappable dual batteries.

FIG. 8 is a flow diagram illustrating a predictive power analytics process executed by a predictive power analytics engine incorporated into the cobot’s control system or, in certain embodiments, a fleet management system. The flowchart of FIG. 8 includes the following steps.

Collect Operational and Battery Data (Step 801). The system continuously collects data from each cobot and its associated batteries. Collected inputs include real-time battery metrics such as state-of-charge trends, discharge rates, number of completed charge cycles, internal resistance measurements, and temperature. The system further collects cobot usage logs indicating performed tasks, task durations, and power consumed per task, as well as environmental data such as operating room temperature. In addition, upcoming surgery schedules or task assignments for each cobot are retrieved to provide insight into future workload.

Model Energy Consumption (Step 802). Using the collected data, the predictive power analytics engine applies one or more energy consumption models, including regression-based models or neural networks, to estimate the amount of energy required for upcoming procedures or tasks. The modeling accounts for task type, expected duration, and which cobot subsystems are anticipated to be active, such as mobility systems, imaging devices, or communication modules. The engine further evaluates battery degradation by comparing predicted performance to observed performance, thereby identifying capacity loss or declining efficiency over time.

Forecast Future State-of-Charge (Step 803). Based on the energy consumption models, the analytics engine forecasts future battery state-of-charge levels at one or more upcoming time points. For example, the system may project a remaining charge percentage at the conclusion of scheduled tasks or procedures. These forecasts are used to determine whether a cobot will have sufficient energy to complete assigned tasks without interruption.

Optimize Charging and Scheduling (Step 804). If the forecasted battery state-of-charge is insufficient for upcoming tasks, the system performs a scheduling optimization to adjust charging plans. This may include modifying fleet-level charging schedules as described with respect to FIG. 6, recommending opportunistic charging during idle periods, or directing replacement or swapping of a battery module as described with respect to FIG. 7. The optimization seeks to ensure adequate energy availability while avoiding excessive facility power demand.

Generate Alerts and Notifications (Step 805). The predictive analytics engine generates one or more alerts when predefined conditions are met. Such alerts may include maintenance alerts indicating that a battery is nearing end-of-life, readiness warnings indicating insufficient predicted capacity to complete an assigned task, fault alerts identifying abnormal battery behavior, or energy shortage alerts indicating excessive concurrent demand across the cobot fleet. Alerts are generated prior to the occurrence of a power-related failure to allow corrective action.

Update Models Through Adaptive Learning (Step 806). After completion of a task or procedure, actual energy consumption data is fed back into the predictive power analytics engine. The engine updates model parameters using machine learning retraining techniques to improve future prediction accuracy. This adaptive learning allows the system to capture cobot-specific usage patterns, procedure-specific variations, and operator-dependent differences over time.

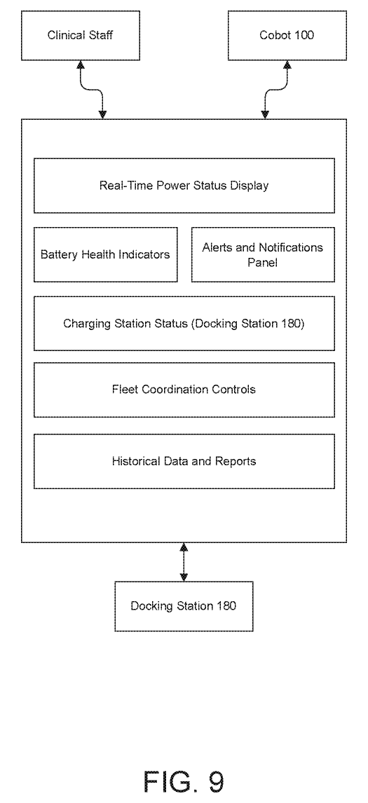

Present Forecasts and Alerts via User Interface (Step 807). The outputs of the predictive analytics process, including energy forecasts, battery health indicators, and generated alerts, are communicated to users through a user interface as described with respect to FIG. 9. The interface may display projected battery depletion times, maintenance recommendations, and readiness warnings, enabling clinical staff to proactively manage charging and battery replacement activities.

Through execution of Steps 801–807, the predictive power analytics process of FIG. 8 provides a closed-loop, machine-learning-driven framework for forecasting energy consumption, coordinating charging activities, and maintaining battery health, thereby improving reliability and reducing unexpected power interruptions in perioperative environments.

FIG. 9 illustrates an example user interface (UI) dashboard that provides hospital staff and operators with real-time information about the cobotic systems’ power status, as well as notifications and controls related to charging and power management. The dashboard is typically displayed on a nurse’s station monitor, tablet, or other control panel accessible in the perioperative area. Consistent with the User Interface and Alert System of the cobotic platform, the UI in FIG. 9 includes several key components:

Real-Time Power Status Display: For each cobot in the fleet, the dashboard prominently shows the current battery charge level (often as a percentage and a graphical battery icon). For example, “Cobot 100: 85% (Fully Charged)” or “Cobot 101: 30% (Charging…)”. Icons may visually resemble conventional battery symbols with color coding – green when charge is healthy, yellow/orange when medium, and red when low. If a cobot is currently docked, an indicator (such as a lightning bolt icon over the battery symbol) shows it is charging. The UI might also display the estimated runtime remaining for each battery, based on current consumption or predictive analytics (e.g., “2.5 hours remaining”).

Battery Health Indicators: In addition to immediate charge, the dashboard can show the long-term health of each battery (state-of-health). This could be a small bar or icon next to the battery level, or accessible by clicking on a cobot. For instance, a cobot’s entry might be colored or marked if its battery health is below a threshold. Detailed info like “Battery Health: 75% of original capacity” or “678/1000 charge cycles used” can be available. This helps plan for battery maintenance or replacement.

Alerts and Notifications Panel: A section of the UI lists active alerts generated by the system (as described in FIG. 8). These could include warnings like “Alert: Cobot 102 Battery Low – 15% remaining” which might flash or turn red if urgent. Other examples: “Cobot 103 requires battery replacement (end-of-life),” or “Charging Station 3 malfunction detected” if a station fails. Alerts are timestamped and may be sorted by priority. The UI ensures that critical alerts (e.g., a cobot in use dropping to critical battery level) are highly visible, possibly accompanied by an audible chime or sent to staff via pager/smartphone as well.

Charging Station Status: The dashboard also shows the status of each docking/charging station 180 in the facility. This could be a simple list or map indicating which stations are occupied, available, or out of service. For an occupied station, it might display which cobot is charging there and how long until charging is complete. This helps staff quickly identify where each robot is and if a station is free in case they need to manually direct a cobot to charge.

Fleet Coordination Controls: Operators can use the UI to override or adjust the autonomous scheduling if necessary. For example, there may be a control to manually dispatch a particular cobot to a specific station (“Dock Cobot 100 at Station 2 now”) or to reserve a charging station for a certain time (perhaps during an expected lull). However, under normal circumstances these decisions are automated; the UI mainly provides visibility. There might also be a way to temporarily pause a cobot’s charging or unplug it via the interface, if, say, an urgent redeployment is needed.

Historical Data and Reports: The dashboard can offer a view into historical power usage, either in a separate tab or pop-up. Graphs could show a cobot’s battery level over the course of a day, or usage vs. charging time for trend analysis. There may be reports that detail power usage per procedure, number of charges per day, etc., which help hospital operations fine-tune how they use the cobots and the infrastructure.

User-Friendly Design: Given the busy environment of an OR, the UI uses clear icons and minimal text for at-a-glance comprehension. For instance, a quick glance might show all cobots as green (good) except one red (needs attention). Each cobot might be represented by a small avatar or number, with a ring or outline indicating battery status. The design leverages familiar symbolism (battery icons, plug icons, warning triangles with exclamation marks for alerts) to ensure that even under stress, the staff can interpret the information quickly.

Security and Access: The UI is likely password-protected or on a secure hospital network, to prevent unauthorized tampering with the robots’ operations. Different user roles can be defined; e.g., biomedical engineers might have access to deeper configuration settings via the UI, whereas nurses see only status and basic controls.

Through the example dashboard of FIG. 9, users are kept in the loop regarding the autonomous power management of the cobot fleet. While the system largely runs on its own, the UI serves as a critical point of transparency and control – showing that all units are charged and healthy, alerting when human intervention is needed, and allowing the healthcare team to feel confident in the cobots’ readiness. This human-machine interface thus complements the automated power management, contributing to overall safety and efficiency in the perioperative environment.

What is claimed is:

1. A perioperative robotic system for automated battery charging and power management, the system comprising:
a mobile robotic device configured for use in a surgical environment, the mobile robotic device including a battery and an integrated power management system configured to monitor one or more parameters of the battery in real time and to dynamically distribute power among a plurality of subsystems of the mobile robotic device;
a docking station comprising a sterility-compliant charging interface configured to couple with the mobile robotic device to recharge the battery outside of a sterile zone; and
wherein the mobile robotic device is configured to autonomously navigate to the docking station for charging while avoiding obstacles and maintaining compliance with sterile-zone restrictions, and wherein the docking station is configured to automatically initiate charging of the battery upon docking of the mobile robotic device.

2. The system of claim 1, wherein the integrated power management system monitors battery parameters including at least voltage, current, temperature, and state-of-health of the battery, and dynamically allocates power among the plurality of subsystems of the mobile robotic device based on real-time operational priorities of the subsystems.

3. The system of claim 1, further comprising an autonomous charging navigation module on the mobile robotic device, wherein the autonomous charging navigation module is configured to map an environment of the mobile robotic device, navigate the mobile robotic device to the docking station while avoiding obstacles, and prevent the mobile robotic device from entering a sterile zone during the navigation.

4. The system of claim 1, wherein the sterility-compliant charging interface comprises a protective enclosure that isolates a charging connection, such that the battery of the mobile robotic device is charged only when the mobile robotic device is outside the sterile zone or inside an enclosed sterile charging bay of the docking station.

5. The system of claim 1, wherein the docking station comprises a communication module configured for bidirectional communication with the mobile robotic device to coordinate docking and automatically initiate charging of the battery, and a multi-port charging architecture configured to allow simultaneous charging of a second mobile robotic device.

6. The system of claim 1, wherein the battery of the mobile robotic device is charged according to an adaptive charging protocol that adjusts charging parameters based on the battery's chemistry, the battery's temperature, and a usage profile of the mobile robotic device.

7. The system of claim 1, further comprising a plurality of mobile robotic devices, wherein the system further comprises a fleet power coordination module configured to prioritize charging among the plurality of mobile robotic devices, balance power loads across one or more docking stations, and prevent charging conflicts at the docking station.

8. The system of claim 1, wherein the mobile robotic device comprises a plurality of modular battery packs that are hot-swappable, an interlock mechanism configured to prevent power interruption during replacement of a battery pack, and an authentication mechanism configured to permit operation only with authorized battery packs.

9. The system of claim 1, further comprising a predictive power analytics engine configured to analyze operational tasks of the mobile robotic device to predict power consumption, and to generate a maintenance alert or a readiness warning when a remaining battery capacity is estimated to be insufficient for completing the predicted tasks.

10. The system of claim 1, further comprising a user interface and alert system configured to display a real-time battery status dashboard and power usage information, to provide alerts for low battery or maintenance conditions, and to enable a user to manually override an automated charging operation.

11. A method for automated battery charging and power management in a perioperative robotic system comprising a mobile robotic device and a docking station, the method comprising:
monitoring one or more battery parameters of a mobile robotic device in real time and dynamically distributing power from a battery of the mobile robotic device among subsystems of the mobile robotic device based on the monitored parameters;
detecting a need to charge the battery of the mobile robotic device;
in response to detecting the need, autonomously navigating the mobile robotic device to a docking station by mapping an environment, avoiding obstacles, and maintaining compliance with sterile-zone restrictions;
docking the mobile robotic device to a sterility-compliant charging interface of the docking station such that the battery is recharged outside of the sterile zone;
initiating charging of the battery upon docking of the mobile robotic device at the docking station; and
regulating the charging of the battery according to an adaptive charging protocol based on a chemistry type of the battery, a temperature of the battery, and a usage profile of the mobile robotic device.

12. The method of claim 11, wherein initiating charging of the battery comprises establishing a bidirectional communication link between the mobile robotic device and the docking station to exchange data for aligning the mobile robotic device with the charging interface and automatically initiating charging.

13. The method of claim 11, wherein the perioperative robotic system comprises a plurality of mobile robotic devices, the method further comprising coordinating charging schedules among the plurality of mobile robotic devices by prioritizing charging for higher-priority devices, balancing power distribution, and avoiding simultaneous docking conflicts at the docking station.

14. The method of claim 11, further comprising detecting a low-battery condition of the mobile robotic device and, in response, replacing a depleted battery pack of the mobile robotic device with a charged battery pack without interrupting power supply to the mobile robotic device.

15. The method of claim 11, further comprising analyzing one or more upcoming tasks of the mobile robotic device to predict future power consumption, and generating an alert when the predicted power consumption exceeds an available battery capacity or when battery maintenance is required.

16. The method of claim 11, further comprising providing, via a user interface, a real-time display of battery status and power management information for the mobile robotic device, issuing alerts for low-battery or maintenance conditions, and enabling a user to manually override an automated charging operation of the mobile robotic device.

17. A non-transitory computer-readable medium storing instructions that, when executed by one or more processors of a perioperative robotic system comprising a mobile robotic device and a docking station, cause the perioperative robotic system to:
monitor one or more parameters of a battery of the mobile robotic device in real time and dynamically distribute power from the battery among subsystems of the mobile robotic device based on the monitored parameters;
detect a need to charge the battery of the mobile robotic device;
autonomously navigate the mobile robotic device to the docking station in response to the detected need by mapping an environment, avoiding obstacles, and complying with sterile-zone restrictions;
dock the mobile robotic device to a sterility-compliant charging interface of the docking station such that the battery is charged outside of the sterile zone;
initiate charging of the battery at the docking station; and
regulate the charging of the battery according to an adaptive charging protocol based on the battery chemistry, the battery temperature, and a usage profile of the mobile robotic device.

18. The non-transitory computer-readable medium of claim 17, wherein the perioperative robotic system comprises a plurality of mobile robotic devices, and the instructions further cause the perioperative robotic system to coordinate charging among the plurality of mobile robotic devices by prioritizing charging requests, balancing power loads across available docking stations, and preventing simultaneous docking conflicts at the docking station.

19. The non-transitory computer-readable medium of claim 17, wherein the instructions further cause the perioperative robotic system to utilize a predictive power analytics engine to forecast power consumption for upcoming tasks of the mobile robotic device and to generate maintenance alerts or readiness warnings when a remaining battery capacity is predicted to be insufficient for the upcoming tasks.

20. The non-transitory computer-readable medium of claim 17, wherein the instructions further cause the perioperative robotic system to provide a user interface that displays a real-time battery status dashboard and power alerts, and that enables a user to manually override an automated charging operation of the mobile robotic device.
